SEQUENCE LISTING (13 sequences)

SEQ ID NO: 1 - DNA, 4593 bp, Mus musculus
  CDS (119)..(4045)
agtttgcctgggtttggcaaggcggagccctggagtaaaacctggggtcggggttgtgagacgcggcccttgcggtcgggcgccagagttgtggggaggacggactgagggccaaagcatgtcctccctccctgggtgcatgagtttggctgcagcgccagctgcagctgactctgcagagattgctgagctgcagcaggcggtggttgaagagctgggtatctctatggaggaacttcgtcagtacattgatgaggaactggaaaagatggactgcatacagcagcgcaagaagcagctcgcagagctggagacgtgggtactacagaaagagtctgaagtggcttatgttgatcggctgtttgatgatgcatccagggaagtgactaactgtgagtctttggtgaaggatttctactctaagctgggactacagtatcatgacagtagctctgaggatgaagcttcccggcccacagagatcattgagattcctgatgaagatgatgatgtcctcagtattgattcaggtgatgctgggagcagaactccaaaagaccagaagcttcgtgaagctatggctgccttaagaaaatcagctcaagatgtccagaagttcatggatgctgtcaacaagaaaagcagttctcaagatctacataaaggaaccttgggtcaggtgtctggagaactgagcaaagatggggacctgatagtcagcatgcggattctgggcaagaagaggactaagacatggcacaaaggcacccttattgccatccagactgttgggctaggaaaaaaatacaaagtgaaatttgacaacaaaggaaagagtctgctatctgggaaccatattgcctatgattaccaccctcccgctgacaagctgtttgtgggcagtcgagtggtggccaagtacaaagatggaaatcaggtctggctttatgctggcattgtagctgagacccctaacgtcaagaacaagctcagatttttaattttttttgatgatggctatgcttcctatgtcactcagtcagagctttatcccatttgccgaccactaaaaaagacttgggaggacatagaagatagctcctgccgagacttcatagaggaatatatcactgcctatccaaaccgcccaatggtacttctcaagagtgggcagcttatcaagactgagtgggaaggcacatggtggaagtctcgagttgaagaggtggatggcagcctagtcaggatcctctttctggatgacaaaagatgtgagtggatatatcgaggctctacacgcctggaacctatgtttagtatgaagacatcctcagcctctgcaatggagaagaagcaaggggggcaactcagaacccgtcctaatatgggtgctgtgaggagcaaaggtcctgttgttcagtatacacaggatctaactggtactggaatccagtttaagcccatggagcccctacagcctatagctccaccggccccacttcctatacctcctctttccccccaagcagctgacactgaaagcttagaaagccaacttgcacaatcacggaaacaagtagccaagaagagcacatcattccgaccaggatctgtgggctccggccattcctcccctacttcatccacactcagtgaaaatgtgtctgctgggaaacttgggataaaccagacatatcggtcacctttggcctcagtaacatctaccccagcatctgcagcccctccagtccctccagtcccaccagggcctccaacccctccagggcctccagctcctccagggcctctagctcctccagccttccatggcatgttagagcgggcaccagctgagccctcctaccgagcccccatggagaagcttttctatttacctcatgtctgcagttacacttgtttgtcccggatcagacccatgagaaacgaacagtatcggggcaagaaccctctattagttccacttctgtatgacttccggaggatgacagcacggcgcagagttaaccgcaaaatgggctttcatgtaatctataagacaccctgtggtctctgccttcggacgatgcaggagatagagcgctacctttttgagactggctgtgactttctgttcctggagatgttctgtttggatccatatgttcttgttgacagaaagtttcaaccctttaagcctttttactatattttggacatcacctatggcaaggaagatgttcccctgtcctgtgttaatgagattgacacaactcccccaccccaggtggcctacagcaaggaacgcattcctggcaagggtgttttcattaacacaggccctgaatttctggttggctgtgactgcaaggatgggtgtcgggataaatccaaatgtgcctgccaccagctaactatccaggccacagcctgtaccccagggggccaagtcaaccctaactctggctaccagtataaaagactagaagagtgtctgcccacaggggtttatgagtgtaacaaacgctgcaattgtgacccaaacatgtgcacaaatcggttggtgcagcatggtctgcaggttcgactacagctgtttaagacacagaacaagggctggggtatccgctgcttggatgatattgccaaaggctcttttgtctgcatttatgcaggcaaaatcctgacagatgactttgcagacaaagaaggcctggagatgggtgatgagtactttgcaaatctggaccacattgaaagtgtggagaacttcaaggaaggatatgagagtgatgtccccacttcctctgacagcagtggggtagatatgaaggaccaggaagatggcaacagcggttcagaggaccctgaagaatccaatgatgacagctctgatgataacttctgtaaggatgaggacttcagcaccagttcagtgtggcgtagctatgctacccggaggcagactcggggtcaaaaggagaatgaattgtctgagatgacttccaaggactcccgccccccagacctcgggcctccacatgttcctatcccttcctcagtatctgtagggggctgcaatccaccttcctctgaagagacacccaagaacaaggtggcctcgtggttgagttgcaatagtgtcagtgaaggtggatttgctgactctgacagccgttcttccttcaagactagtgaaggtggagatggccgtgctgggggaggccggggagaggctgaaagggcctctacctcaggattgagcttcaaggatgaaggagacaataagcagcctaaaaaagaggaccctgagaaccgaaacaagatgccagtagttactgaaggctctcagaatcatggacataatcctcccatgaagtctgaagggcttcgccgaccagctagtaaaatgtctgtgctccagagccagcgagttgtgacttctactcagtcaaaccctgatgacatcctgacactgtccagcagcacagagagtgagggggaaagtggaaccagccgaaagcccactgctggtcacacttcagccacagctgttgatagtgatgacatccagaccatctcttctggctctgacggtgatgactttgaggacaagaagaacttgtcaggaccaacaaagcgccaggtggcagtaaaatcaacccgaggctttgctcttaaatcaacccatggtattgccattaaatcaaccaacatggcttccgtggacaagggggagagtgcaccagttcgtaagaacacacgccagttctatgatggtgaagagtcttgctacatcattgatgccaaacttgaaggcaacctaggccgctacctcaatcacagttgcagccccaacctgtttgtccagaatgtgtttgtggatacccatgatcttcgcttcccttgggtggccttctttgccagcaagagaatccgggctggaacagaactcacttgggactacaactacgaagtgggcagtgtggaaggcaaggagctgctgtgctgctgtggggccattgaatgcagagggagacttctttagaggaagacttcctcactttgagaacgcttgaactatccttttccccaggaactgggtcttcctgactgttgaactctgaccccaagtctctgatctagcttcttcccagctcctagtagatagagatggggattctcaatcaggactttcccagcgtggtgctagcaggcactagggtgggtagacatgaccactctagcatcagcctgaggtccttctcatcttgtatgcattcatgattcacatgggctgtgtatctgccatccctgatttgtatggtttcttgaaagtcttctaacaagatggtaaagtagagattgtggtttgtcttatctcctgccttgatcttccatgtcatttcccagtgggcaactatcattagtttatacctcgattttatttgccctgtgtaatttttaactaaaaatgttacataaccacaagggaggcctaagaaaaaaactaaccctttctcaagtcttaatagtctgacacagtcatgttttatttaacctttaatatactttaattaaaaaaaattaacagta

SEQ ID NO: 2 - protein, 1308 aa, Mus musculus
MSSLPGCMSLAAAPAAADSAEIAELQQAVVEELGISMEELRQYIDEELEKMDCIQQRKKQLAELETWVLQKESEVAYVDRLFDDASREVTNCESLVKDFYSKLGLQYHDSSSEDEASRPTEIIEIPDEDDDVLSIDSGDAGSRTPKDQKLREAMAALRKSAQDVQKFMDAVNKKSSSQDLHKGTLGQVSGELSKDGDLIVSMRILGKKRTKTWHKGTLIAIQTVGLGKKYKVKFDNKGKSLLSGNHIAYDYHPPADKLFVGSRVVAKYKDGNQVWLYAGIVAETPNVKNKLRFLIFFDDGYASYVTQSELYPICRPLKKTWEDIEDSSCRDFIEEYITAYPNRPMVLLKSGQLIKTEWEGTWWKSRVEEVDGSLVRILFLDDKRCEWIYRGSTRLEPMFSMKTSSASAMEKKQGGQLRTRPNMGAVRSKGPVVQYTQDLTGTGIQFKPMEPLQPIAPPAPLPIPPLSPQAADTESLESQLAQSRKQVAKKSTSFRPGSVGSGHSSPTSSTLSENVSAGKLGINQTYRSPLASVTSTPASAAPPVPPVPPGPPTPPGPPAPPGPLAPPAFHGMLERAPAEPSYRAPMEKLFYLPHVCSYTCLSRIRPMRNEQYRGKNPLLVPLLYDFRRMTARRRVNRKMGFHVIYKTPCGLCLRTMQEIERYLFETGCDFLFLEMFCLDPYVLVDRKFQPFKPFYYILDITYGKEDVPLSCVNEIDTTPPPQVAYSKERIPGKGVFINTGPEFLVGCDCKDGCRDKSKCACHQLTIQATACTPGGQVNPNSGYQYKRLEECLPTGVYECNKRCNCDPNMCTNRLVQHGLQVRLQLFKTQNKGWGIRCLDDIAKGSFVCIYAGKILTDDFADKEGLEMGDEYFANLDHIESVENFKEGYESDVPTSSDSSGVDMKDQEDGNSGSEDPEESNDDSSDDNFCKDEDFSTSSVWRSYATRRQTRGQKENELSEMTSKDSRPPDLGPPHVPIPSSVSVGGCNPPSSEETPKNKVASWLSCNSVSEGGFADSDSRSSFKTSEGGDGRAGGGRGEAERASTSGLSFKDEGDNKQPKKEDPENRNKMPVVTEGSQNHGHNPPMKSEGLRRPASKMSVLQSQRVVTSTQSNPDDILTLSSSTESEGESGTSRKPTAGHTSATAVDSDDIQTISSGSDGDDFEDKKNLSGPTKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKRIRAGTELTWDYNYEVGSVEGKELLCCCGAIECRGRLL

SEQ ID NO: 3 - DNA, 4449 bp, Homo sapiens
  CDS (191)..(4066)
ggcactaaaggtttgcttccgggcgtttcttttgcttccccttccctctttcacgcttcctcccctccccctcctcccttatcccttcgctttcgctcttttccgtcgaggccgacccctgagttgtgagtctggggtctggttggtgaaaaagagcccttgaagctggaagacgggagaggacaaaagcatgtcttcccttcctgggtgcattggtttggatgcagcaacagctacagtggagtctgaagagattgcagagctgcaacaggcagtggttgaggaactgggtatctctatggaggaacttcggcatttcatcgatgaggaactggagaagatggattgtgtacagcaacgcaagaagcagctagcagagttagagacatgggtaatacagaaagaatctgaggtggctcacgttgaccaactctttgatgatgcatccagggcagtgactaattgtgagtctttggtgaaggacttctactccaagctgggactacaataccgggacagtagctctgaggacgaatcttcccggcctacagaaataattgagattcctgatgaagatgatgatgtcctcagtattgattcaggtgatgctgggagcagaactccaaaagaccagaagctccgtgaagctatggctgccttaagaaagtcagctcaagatgttcagaagttcatggatgctgtcaacaagaagagcagttcccaggatctgcataaaggaaccttgagtcagatgtctggagaactaagcaaagatggtgacctgatagtcagcatgcgaattctgggcaagaagagaactaagacttggcacaaaggcacccttattgccatccagacagttgggccagggaagaaatacaaggtgaaatttgacaacaaaggaaagagtctactgtcggggaaccatattgcctatgattaccaccctcctgctgacaagctgtatgtgggcagtcgggtggtcgccaaatacaaagatgggaatcaggtctggctctatgctggcattgtagctgagacaccaaacgtcaaaaacaagctcaggtttctcattttctttgatgatggctatgcttcctatgtcacacagtcggaactgtatcccatttgccggccactgaaaaagacttgggaggacatagaagacatctcctgccgtgacttcatagaggagtatgtcactgcctaccccaaccgccccatggtactgctcaagagtggccagcttatcaagactgagtgggaaggcacgtggtggaagtcccgagttgaggaggtggatggcagcctagtcaggatcctcttcctggatgacaaaagatgtgagtggatctatcgaggctctacacggctggagcccatgttcagcatgaaaacatcctcagcctctgcactggagaagaagcaaggacagctcaggacacgtccaaatatgggtgctgtgaggagcaaaggccctgttgtccagtacacacaggatctgaccggtactggaacccagttcaagccagtggaacccccacagcctacagctccacctgccccacctttcccacctgctccacctctatccccccaagcaggtgacagtgacttggaaagccagcttgcccagtcacggaagcaggtagccaaaaagagcacgtcctttcgaccaggatctgtgggctctggtcattcctcccctacatctcctgcactcagtgaaaatgtctctggtgggaaacctgggatcaaccagacatatagatcacctttaggctccacagcctctgccccagcaccctcagcactcccggcccctccagcacccccagtcttccatggcatgctggagcgggccccagcagagccctcctaccgtgctcccatggagaagcttttctacttacctcatgtctgcagctatacctgtctgtctcgagtcagacctatgaggaatgagcagtaccggggcaagaaccctctgctggtcccgttactatatgacttccggcggatgacagcccggcgtcgagttaaccgcaagatgggctttcatgttatctataagacaccttgtggtctctgccttcggacaatgcaggagatagaacgctaccttttcgagactggctgtgacttcctcttcctggagatgttctgtttggatccatatgttcttgtggaccgaaagtttcagccctataagcctttttactatattttggacatcacttatgggaaggaagatgttcccctatcctgtgtcaatgagattgacacaacccctccaccccaggtggcctacagcaaggaacgtatcccgggcaagggtgttttcattaacacaggccctgaatttctggttggctgtgactgcaaggatgggtgtcgggacaagtccaagtgtgcctgccatcaactaactatccaggctacagcctgtaccccaggaggccaaatcaaccctaactctggctaccagtacaagagactagaagagtgtctacccacaggggtatatgagtgtaacaaacgctgcaaatgtgacccaaacatgtgcacaaaccggttggtgcaacatggactacaagttcggctacagctattcaagacacagaacaagggctggggtatccgctgcttggatgacattgccaaaggctcttttgtttgtatttatgcaggcaaaatcctgacagatgactttgcagacaaggagggtctggaaatgggtgatgagtactttgcaaatctggaccatatcgagagcgtggagaacttcaaagaaggatatgagagtgatgccccctgttcctctgacagcagtggtgtagacttgaaggaccaggaagatggcaacagcggtacagaggaccctgaagagtccaatgatgatagctcagatgataacttctgtaaggatgaggacttcagcaccagttcagtgtggcggagctatgctacccggaggcagacccggggccagaaagagaacggactctctgagacaacttccaaggactcccaccccccagatcttggacccccacatattcctgttcctccctcaatccctgtaggtggctgcaatccaccttcctccgaagagacacccaagaacaaggtggcctcatggttgagctgcaatagtgtcagtgaaggtggttttgctgactctgatagccattcatccttcaagactaatgaaggtggggagggccgggctgggggaagccgaatggaggctgagaaggcctccacctcaggactaggcatcaaggatgagggagacatcaaacaggccaagaaagaggacactgacgaccgaaacaagatgtcagtagttactgaaagctctcgaaattacggttacaatccttctcctgtgaagcctgaaggacttcgccgcccacctagtaagactagtatgcatcaaagccgaagactcatggcttctgctcagtccaaccctgatgatgtcctgacactgtccagcagcacagaaagtgagggggaaagtgggaccagccgaaagcccactgctggtcagacttcggctacagcggttgacagtgatgatatccagaccatatcctctggctctgaaggggatgactttgaggacaagaagaacatgactggtccaatgaagcgtcaagtggcagtaaaatcaacccgaggctttgctcttaaatcaacccatgggattgcaattaaatcaaccaacatggcctctgtggacaagggggagagcgcacctgttcgtaagaacacacgccaattctatgatggcgaggagtcttgctacatcattgatgccaagcttgaaggcaacctgggccgctacctcaaccacagttgcagccccaacctgtttgtccagaatgtcttcgtggatacccatgatcttcgcttcccctgggtggccttctttgccagcaaaagaatccgggctgggacagaacttacttgggactacaactacgaggtgggcagtgtggaaggcaaggagctactctgttgctgtggggccattgaatgcagaggacgtcttctttagaggacagccttcttcccaacccttcttgaactgtcgtttcctcaggaactgggtcttcctgattgttgaaccctgacccgaagtctctgggctagctactccccccagctcctagttgatagaaatgggggttctggaccagatgatcccttccaatgtggtgctagcaggcaggatcccttctccacctccaaaggccctaaagggtggggagagatcaccactctaacctcggcctgacatccctcccatcccatatttgtccaagtgttcctgcttctaacagactttgttcttagaatggagcctgtgtatctactatctccagtttgtattatttcttgaaagtcttttaacaatatgataaaactaagattgtgaaa

SEQ ID NO: 4 - protein, 1291 aa, Homo sapiens
MSSLPGCIGLDAATATVESEEIAELQQAVVEELGISMEELRHFIDEELEKMDCVQQRKKQLAELETWVIQKESEVAHVDQLFDDASRAVTNCESLVKDFYSKLGLQYRDSSSEDESSRPTEIIEIPDEDDDVLSIDSGDAGSRTPKDQKLREAMAALRKSAQDVQKFMDAVNKKSSSQDLHKGTLSQMSGELSKDGDLIVSMRILGKKRTKTWHKGTLIAIQTVGPGKKYKVKFDNKGKSLLSGNHIAYDYHPPADKLYVGSRVVAKYKDGNQVWLYAGIVAETPNVKNKLRFLIFFDDGYASYVTQSELYPICRPLKKTWEDIEDISCRDFIEEYVTAYPNRPMVLLKSGQLIKTEWEGTWWKSRVEEVDGSLVRILFLDDKRCEWIYRGSTRLEPMFSMKTSSASALEKKQGQLRTRPNMGAVRSKGPVVQYTQDLTGTGTQFKPVEPPQPTAPPAPPFPPAPPLSPQAGDSDLESQLAQSRKQVAKKSTSFRPGSVGSGHSSPTSPALSENVSGGKPGINQTYRSPLGSTASAPAPSALPAPPAPPVFHGMLERAPAEPSYRAPMEKLFYLPHVCSYTCLSRVRPMRNEQYRGKNPLLVPLLYDFRRMTARRRVNRKMGFHVIYKTPCGLCLRTMQEIERYLFETGCDFLFLEMFCLDPYVLVDRKFQPYKPFYYILDITYGKEDVPLSCVNEIDTTPPPQVAYSKERIPGKGVFINTGPEFLVGCDCKDGCRDKSKCACHQLTIQATACTPGGQINPNSGYQYKRLEECLPTGVYECNKRCKCDPNMCTNRLVQHGLQVRLQLFKTQNKGWGIRCLDDIAKGSFVCIYAGKILTDDFADKEGLEMGDEYFANLDHIESVENFKEGYESDAPCSSDSSGVDLKDQEDGNSGTEDPEESNDDSSDDNFCKDEDFSTSSVWRSYATRRQTRGQKENGLSETTSKDSHPPDLGPPHIPVPPSIPVGGCNPPSSEETPKNKVASWLSCNSVSEGGFADSDSHSSFKTNEGGEGRAGGSRMEAEKASTSGLGIKDEGDIKQAKKEDTDDRNKMSVVTESSRNYGYNPSPVKPEGLRRPPSKTSMHQSRRLMASAQSNPDDVLTLSSSTESEGESGTSRKPTAGQTSATAVDSDDIQTISSGSEGDDFEDKKNMTGPMKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKRIRAGTELTWDYNYEVGSVEGKELLCCCGAIECRGRLL

SEQ ID NO: 5 - DNA, 4446 bp, HOMO SAPIENS
  CDS (191)..(4063)
ggcactaaaggtttgcttccgggcgtttcttttgcttccccttccctctttcacgcttcctcccctccccctcctcccttatcccttcgctttcgctcttttccgtcgaggccgacccctgagttgtgagtctggggtctggttggtgaaaaagagcccttgaagctggaagacgggagaggacaaaagcatgtcttcccttcctgggtgcattggtttggatgcagcaacagctacagtggagtctgaagagattgcagagctgcaacaggcagtggttgaggaactgggtatctctatggaggaacttcggcatttcatcgatgaggaactggagaagatggattgtgtacagcaacgcaagaagcagctagcagagttagagacatgggtaatacagaaagaatctgaggtggctcacgttgaccaactctttgatgatgcatccagggcagtgactaattgtgagtctttggtgaaggacttctactccaagctgggactacaataccgggacagtagctctgaggacgaatcttcccggcctacagaaataattgagattcctgatgaagatgatgatgtcctcagtattgattcaggtgatgctgggagcagaactccaaaagaccagaagctccgtgaagctatggctgccttaagaaagtcagctcaagatgttcagaagttcatggatgctgtcaacaagaagagcagttcccaggatctgcataaaggaaccttgagtcagatgtctggagaactaagcaaagatggtgacctgatagtcagcatgcgaattctgggcaagaagagaactaagacttggcacaaaggcacccttattgccatccagacagttgggccagggaagaaatacaaggtgaaatttgacaacaaaggaaagagtctactgtcggggaaccatattgcctatgattaccaccctcctgctgacaagctgtatgtgggcagtcgggtggtcgccaaatacaaagatgggaatcaggtctggctctatgctggcattgtagctgagacaccaaacgtcaaaaacaagctcaggtttctcattttctttgatgatggctatgcttcctatgtcacacagtcggaactgtatcccatttgccggccactgaaaaagacttgggaggacatagaagacatctcctgccgtgacttcatagaggagtatgtcactgcctaccccaaccgccccatggtactgctcaagagtggccagcttatcaagactgagtgggaaggcacgtggtggaagtcccgagttgaggaggtggatggcagcctagtcaggatcctcttcctggatgacaaaagatgtgagtggatctatcgaggctctacacggctggagcccatgttcagcatgaaaacatcctcagcctctgcactggagaagaagcaaggacagctcaggacacgtccaaatatgggtgctgtgaggagcaaaggccctgttgtccagtacacacaggatctgaccggtactggaacccagttcaagccagtggaacccccacagcctacagctccacctgccccacctttcccacctgctccacctctatccccccaagcaggtgacagtgacttggaaagccagcttgcccagtcacggaagcaggtagccaaaaagagcacgtcctttcgaccaggatctgtgggctctggtcattcctcccctacatctcctgcactcagtgaaaatgtctctggtgggaaacctgggatcaaccagacatatagatcacctttaggctccacagcctctgccccagcaccctcagcactcccggcccctccagcacccccagtcttccatggcatgctggagcgggccccagcagagccctcctaccgtgctcccatggagaagcttttctacttacctcatgtctgcagctatacctgtctgtctcgagtcagacctatgaggaatgagcagtaccggggcaagaaccctctgctggtcccgttactatatgacttccggcggatgacagcccggcgtcgagttaaccgcaagatgggctttcatgttatctataagacaccttgtggtctctgccttcggacaatgcaggagatagaacgctaccttttcgagactggctgtgacttcctcttcctggagatgttctgtttggatccatatgttcttgtggaccgaaagtttcagccctataagcctttttactatattttggacatcacttatgggaaggaagatgttcccctatcctgtgtcaatgagattgacacaacccctccaccccaggtggcctacagcaaggaacgtatcccgggcaagggtgttttcattaacacaggccctgaatttctggttggctgtgactgcaaggatgggtgtcgggacaagtccaagtgtgcctgccatcaactaactatccaggctacagcctgtaccccaggaggccaaatcaaccctaactctggctaccagtacaagagactagaagagtgtctacccacaggggtatatgagtgtaacaaacgctgcaaatgtgacccaaacatgtgcacaaaccggttggtgcaacatggactacaagttcggctacagctattcaagacacagaacaagggctggggtatccgctgcttggatgacattgccaaaggctcttttgtttgtatttatgcaggcaaaatcctgacagatgactttgcagacaaggagggtctggaaatgggtgatgagtactttgcaaatctggaccatatcgagagcgtggagaacttcaaagaaggatatgagagtgatgccccctgttcctctgacagcagtggtgtagacttgaaggaccaggaagatggcaacagcggtacagaggaccctgaagagtccaatgatgatagctcagatgataacttctgtaaggatgaggacttcagcaccagttcagtgtggcggagctatgctacccggaggcagacccggggccagaaagagaacggactctctgagacaacttccaaggactcccaccccccagatcttggacccccacatattcctgttcctccctcaatccctgtaggtggctgcaatccaccttcctccgaagagacacccaagaacaaggtggcctcatggttgagctgcaatagtgtcagtgaaggtggttttgctgactctgatagccattcatccttcaagactaatgaaggtggggagggccgggctgggggaagccgaatggaggctgagaaggcctccacctcaggactaggcatcaaggatgagggagacatcaaacaggccaagaaagaggacactgacgaccgaaacaagatgtcagtagttactgaaagctctcgaaattacggttacaatccttctcctgtgaagcctgaaggacttcgccgcccacctagtaagactagtatgcatcaaagccgaagactcatggcttctgctcagtccaaccctgatgatgtcctgacactgtccagcagcacagaaagtgagggggaaagtgggaccagccgaaagcccactgctggtcagacttcggctacagcggttgacagtgatgatatccagaccatatcctctggctctgaaggggatgactttgaggacaagaagaacatgactggtccaatgaagcgtcaagtggcagtaaaatcaacccgaggctttgctcttaaatcaacccatgggattgcaattaaatcaaccaacatggcctctgtggacaagggggagagcgcacctgttcgtaagaacacacgccaattctatgatggcgaggagtcttgctacatcattgatgccaagcttgaaggcaacctgggccgctacctcaaccacagttgcagccccaacctgtttgtccagaatgtcttcgtggatacccatgatcttcgcttcccctgggtggccttctttgccagcaaaatccgggctgggacagaacttacttgggactacaactacgaggtgggcagtgtggaaggcaaggagctactctgttgctgtggggccattgaatgcagaggacgtcttctttagaggacagccttcttcccaacccttcttgaactgtcgtttcctcaggaactgggtcttcctgattgttgaaccctgacccgaagtctctgggctagctactccccccagctcctagttgatagaaatgggggttctggaccagatgatcccttccaatgtggtgctagcaggcaggatcccttctccacctccaaaggccctaaagggtggggagagatcaccactctaacctcggcctgacatccctcccatcccatatttgtccaagtgttcctgcttctaacagactttgttcttagaatggagcctgtgtatctactatctccagtttgtattatttcttgaaagtcttttaacaatatgataaaactaagattgtgaaa

SEQ ID NO: 6 - protein, 1290 aa, HOMO SAPIENS
MSSLPGCIGLDAATATVESEEIAELQQAVVEELGISMEELRHFIDEELEKMDCVQQRKKQLAELETWVIQKESEVAHVDQLFDDASRAVTNCESLVKDFYSKLGLQYRDSSSEDESSRPTEIIEIPDEDDDVLSIDSGDAGSRTPKDQKLREAMAALRKSAQDVQKFMDAVNKKSSSQDLHKGTLSQMSGELSKDGDLIVSMRILGKKRTKTWHKGTLIAIQTVGPGKKYKVKFDNKGKSLLSGNHIAYDYHPPADKLYVGSRVVAKYKDGNQVWLYAGIVAETPNVKNKLRFLIFFDDGYASYVTQSELYPICRPLKKTWEDIEDISCRDFIEEYVTAYPNRPMVLLKSGQLIKTEWEGTWWKSRVEEVDGSLVRILFLDDKRCEWIYRGSTRLEPMFSMKTSSASALEKKQGQLRTRPNMGAVRSKGPVVQYTQDLTGTGTQFKPVEPPQPTAPPAPPFPPAPPLSPQAGDSDLESQLAQSRKQVAKKSTSFRPGSVGSGHSSPTSPALSENVSGGKPGINQTYRSPLGSTASAPAPSALPAPPAPPVFHGMLERAPAEPSYRAPMEKLFYLPHVCSYTCLSRVRPMRNEQYRGKNPLLVPLLYDFRRMTARRRVNRKMGFHVIYKTPCGLCLRTMQEIERYLFETGCDFLFLEMFCLDPYVLVDRKFQPYKPFYYILDITYGKEDVPLSCVNEIDTTPPPQVAYSKERIPGKGVFINTGPEFLVGCDCKDGCRDKSKCACHQLTIQATACTPGGQINPNSGYQYKRLEECLPTGVYECNKRCKCDPNMCTNRLVQHGLQVRLQLFKTQNKGWGIRCLDDIAKGSFVCIYAGKILTDDFADKEGLEMGDEYFANLDHIESVENFKEGYESDAPCSSDSSGVDLKDQEDGNSGTEDPEESNDDSSDDNFCKDEDFSTSSVWRSYATRRQTRGQKENGLSETTSKDSHPPDLGPPHIPVPPSIPVGGCNPPSSEETPKNKVASWLSCNSVSEGGFADSDSHSSFKTNEGGEGRAGGSRMEAEKASTSGLGIKDEGDIKQAKKEDTDDRNKMSVVTESSRNYGYNPSPVKPEGLRRPPSKTSMHQSRRLMASAQSNPDDVLTLSSSTESEGESGTSRKPTAGQTSATAVDSDDIQTISSGSEGDDFEDKKNMTGPMKRQVAVKSTRGFALKSTHGIAIKSTNMASVDKGESAPVRKNTRQFYDGEESCYIIDAKLEGNLGRYLNHSCSPNLFVQNVFVDTHDLRFPWVAFFASKIRAGTELTWDYNYEVGSVEGKELLCCCGAIECRGRLL

SEQ ID NO: 7 - DNA, 58 bp, Artificial Sequence
  shRNA sequence
ccgggccttgatcttccatgtcattctcgagaatgacatggaagatcaaggctttttg

SEQ ID NO: 8 - DNA, 58 bp, Artificial Sequence
  shRNA sequence
ccggcccatgagaaacgaacagtatctcgagatactgttcgtttctcatgggtttttg

SEQ ID NO: 9 - DNA, 58 bp, Artificial Sequence
  shRNA sequence
ccggcccgaggctttgctcttaaatctcgagatttaagagcaaagcctcgggtttttg

SEQ ID NO: 10 - DNA, 58 bp, Artificial Sequence
  shRNA sequence
ccggccacattgaaagtgtggagaactcgagttctccacactttcaatgtggtttttg

SEQ ID NO: 11 - DNA, 58 bp, Artificial Sequence
  shRNA sequence
ccggccagacatatcggtcacctttctcgagaaaggtgaccgatatgtctggtttttg

SEQ ID NO: 12 - DNA, 60 bp, Artificial Sequence
  shRNA sequence
gatccccgatgtgagtggatatatcgttcaagagacgatatatccactcacatcttttta

SEQ ID NO: 13 - DNA, 60 bp, Artificial Sequence
  shRNA sequence
agcttaaaaagatgtgagtggatatatcgtctcttgaacgatatatccactcacatcggg

(19) United States
(12) Patent Application Publication
(10) Pub. No.: US 2011/0190152 A1
(43) Pub. Date: Aug. 4, 2011

(54) PLURIPOTENCY ASSOCIATED EPIGENETIC FACTOR

(76) Inventors: Leng-Siew Yeap, Cambridge (GB); Katsuhiko Hayashi, Cambridge (GB); Azim Surani, Cambridge (GB)

(21) Appl. No.: 12/995,824

(22) PCT Filed: Jun. 4, 2009
(86) PCT No.: PCT/GB2009/050628

§ 371 Date: Apr. 13, 2011

(30) Foreign Application Priority Data
Jun. 4, 2008 (GB) 0810209.7

(51) Int. Cl. C40B 30/04; C12N 5/071; C12N 15/85; C12N 5/10; C12M 3/00; C07H 21/02; C07H 21/00; C40B 30/00

(52) U.S. Cl. 506/9; 435/375; 435/455; 435/325; 435/289.1; 435/463; 435/366; 536/24.5; 506/7

(57) ABSTRACT

A method for controlling the pluripotent phenotype of a cell comprising modulating the expression or activity of a ESET/SETDB1 polypeptide, or a homologue thereof, within the cell is provided. Pluripotent cells, cultures of such cells and methods for reprogramming somatic cells to a pluripotent phenotype comprising expressing a ESET/SETDB1 polypeptide in the cells, either alone or in combination with other pluripotency factors, are further provided. Methods for identifying modulators of pluripotency and their use in treating cancer or cancer stem cells are also provided.

### FIELD

The invention relates to cellular factors involved in reprogramming of cells and cell nuclei to adopt a pluripotent state, as well as factors that maintain that pluripotent state. The invention also relates to identification of agents that modulate the epigenetic activity of pluripotency associated factors in vitro and in vivo.

### BACKGROUND

Somatic cells typically develop along a differentiation pathway progressing from a less specialised to a more specialised or committed state. Less specialised somatic cells can demonstrate the ability to act as progenitor stem cells giving rise to several different cell types. The amount of these different cell types that a given stem cell can act as a progenitor for is typically referred to as the ‘potency’ of that stem cell. Pluripotent stem cells can act as progenitors for very many different differentiated cell types. If a cell can differentiate into all cells in the body, it is considered to be totipotent. If it can differentiate into most cell types, it is pluripotent. Embryonic stem (ES) cells are usually referred to as pluripotent as they are capable of self-renewal and can generate most cell types in mammals with the exception of extra-embryonic tissues (i.e. trophectoderm). A number of pluripotent cell types are known in addition to ES cells, including embryonal carcinoma (EC) cells, induced pluripotent stem cells (iPS cells), epiblast stem cells (EpiSCs), embryonic germ (EG) cells and primordial germ cells (PGCs).

The homeodomain containing transcription factors Oct4 (POU5F1) and Nanog have been identified as essential regulators of ES cell identity and are, thus, considered to be important in the maintenance of pluripotency (Nichols et al. Cell (1998) 95: 379-391; and Chambers et al. Cell (2003) 113: 643-655). One of the key challenges in stem cell biology is identifying the mechanism of action by which these pluripotency associated transcription factors control the epigenetic status of the pluripotent cell. Indeed, it is the epigenome of a given cell that ultimately determines whether it can become pluripotent, and as a result attempts to reprogram somatic cell nuclei into pluripotent cell nuclei rely on the presence of factors that can modify the epigenome of these cells into that of an ES-like cell. Large scale epigenetic reprogramming occurs in mammalian germ cells and the early embryo. Epigenetic reprogramming in the germline and early embryo is, therefore, crucial for maintaining the pluripotency of germ and embryonic stem cells. This reprogramming involves controlling widespread chemical modifications of both the genomic DNA and also the multitude of proteins associated with DNA which together interact to form chromatin. It is these changes that in turn regulate the expression of the genes that determine the phenotype of the cell. Pluripotent stem cells are of great value in fields such as regenerative medicine where they can serve as progenitors for cells and tissues that can be used in treating degenerative diseases, cell therapy, treatment of trauma and generally in the replacement of worn out organs. Pluripotent stem cells are also of value in drug screening assays, as they can provide a source of human tissue types in vitro thereby abrogating the need for extensive animal testing. Pluripotent stem cells, such as ES cells, are also key to the production of transgenic animals.

In humans there has been significant controversy around the use of human ES cells, which until recently could only be obtained from early stage human embryos. The desire to seek alternative sources of human pluripotent cells lead researchers to express key pluripotency determining factors ectopically (including Oct4) within differentiated somatic cells in order to ‘reprogram’ these cells into assuming a pluripotent phenotype (Okita K, Ichisaka T, Yamanaka S. Nature (2007) 448: 313-7). These experiments demonstrate the basic principle that ectopic expression of a group of pluripotency associated genes can lead to apparent cellular reprogramming, however the actual mechanism by which these factors exert their reprogramming effects remains unknown. Clearly if therapies are to be based upon these advances more needs to be understood about the cellular mechanisms by which the pluripotent state is achieved and controlled.

Since stem cells posses the combined abilities to both extensively self-renew and differentiate into progenitors they are also potential candidates for the origin of many cancers (Beachy et al. Nature (2004) 432:324-31). Stem cells can have a long lifespan in which they acquire genetic mutations and epigenetic modifications that can increase the tendency toward malignancy. It is postulated that since stem cells occupy a niche that is so finely balanced between the competing interests of proliferation and differentiation, small but profound epigenetic changes can tip the balance towards a cancer stem cell phenotype. An appreciation of why and how epigenetic modifications are regulated is critical to the understanding, detection and treatment of cancer and particularly the treatment of cancer stem cells. Indeed, it is believed that one of the factors present in cases of recurrent and aggressive cancers that are difficult to treat is that the tumours may contain cancer stem cells that do not respond well or at all to conventional therapies.

### SUMMARY

The present invention is based in part upon the characterisation of the interaction between the pluripotency transcription factor Oct4 and the epigenetic modifying enzyme ESET/SETDB1. Accordingly, a first aspect of the invention provides a method for controlling the pluripotent phenotype of a cell comprising modulating the expression or activity of a ESET/SETDB1 polypeptide, or a homologue thereof, within the cell. Activity of the ESET/SETDB1 polypeptide is typically modulated by exposing the cell to a compound or molecule that modulates the catalytic activity of ESET/SETDB1. Such a compound or molecule may be selected from: a small molecule, an aptamer, a polypeptide, and oligopeptide, an oligonucleotide, a polyamine, an analogue of s-adenosyl-methionine, a substituted form of s-adenosyl-methionine, a nucleotide analogue, a nucleoside analogue, or an antibody or a fragment thereof. Optionally the compound or molecule is an inhibitor of ESET/SETDB1 catalytic activity, thereby promoting differentiation of a pluripotent cell. Alternatively, the compound or molecule agonises or promotes ESET/SETDB1 catalytic activity, thereby inhibiting differentiation and promoting self renewal of the pluripotent phenotype.

In one embodiment of the invention an ESET/SETDB1 encoding polynucleotide sequence is introduced into the cell and expressed via a heterologous expression vector. Suitably, the heterologous expression vector is an episomal vector. The heterologous expression vector can comprise a nucleic acid sequence that encodes an ESET/SETDB1 polypeptide that is operatively linked to a promoter sequence. The promoter sequence may comprise an inducible promoter or a constitutively active promoter, depending on the particular requirement for expression in the cell. Optionally, the promoter sequence can comprise at least one sequence element that is capable of binding a pluripotency associated transcription factor (for example, Oct4 or nanog).

In a further specific embodiment of the invention, the heterologous expression vector integrates into the genome of the cell via homologous recombination. In this embodiment the expression vector may comprise a promoter sequence that is operatively linked to the ESET/SETDB1 nucleic acid sequence. Alternatively, the expression vector may lack a promoter sequence and rely on the presence of an endogenous promoter located close to or at the site of integration into the host cell genome in order to initiate ESET/SETDB1 expression in vivo.

In a further specific embodiment, the invention provides for modulation of expression of the ESET/SETDB1 polypeptide in the cell by exposing the cell to a compound selected from: an siRNA, an shRNA, an antisense oligonucleotide, or an antisense polynucleotide. Suitably, the compound comprises an shRNA selected from one or more of SEQ ID NOs: 3-9.

The abovementioned embodiments of the invention that provide for expression or agonisation of an ESET/SETDB1 activity within the cell may optionally be for the purpose of inducing reprogramming of the cell into a more pluripotent phenotype. Alternatively, the purpose can be to prevent differentiation of a pluripotent cell and/or promote propagation of the pluripotent phenotype—for example, within a culture of pluripotent stem cells.

A second aspect of the invention provides a mammalian cell comprising a heterologous expression vector that encodes an ESET/SETDB1 polypeptide, or a homologue or derivative thereof. In an embodiment of the invention the heterologous expression vector is integrated into the genome of the cell via homologous recombination. In another embodiment of the invention, the heterologous expression vector is episomally maintained. Optionally, the cell is selected from: a somatic cell, a multipotent stem cell, a unipotent stem cell, a cancer cell, a cancer cell line cell, and a pluripotent cell. The cell may suitably be a human cell, with the proviso that it is not a cell that has been obtained directly from a human embryo. In a specific embodiment of the invention, the heterologous expression vector comprises a promoter in operative combination with a nucleic acid sequence that encodes the ESET/SETDB1 polypeptide. Optionally, the cells may be in the form of a composition or kit, such as a lyophilised or vitrified composition. The invention also provides for a culture vessel comprising a culture of pluripotent mammalian stem cells obtained according to the aforementioned methods and a culture medium suitable for maintaining the pluripotent stem cells.

In a third aspect the invention provides a method for reprogramming a somatic cell nucleus comprising expressing ESET/SETDB1 polypeptide, or a homologue thereof, in a somatic cell that comprises the nucleus in combination with one or more pluripotency associated transcription factors. Optionally, the pluripotency associated transcription factor is selected from one or more of the group consisting of Oct3, Oct4, nanog, sox2, c-myc and klf4 (sometimes called the ‘Yamanaka factors’) or additional factors such as Dppa3/4. The somatic cell nucleus is suitably obtained from: a multipotent stem cell, a unipotent stem cell, a germ cell and a terminally differentiated cell. The cell may suitably be a human cell. In a specific embodiment of the invention the method further comprises exposing the cell to an inhibitor of the MEK/ERK signalling pathway.

A fourth aspect of the invention provides for an isolated polypeptide complex comprising at least a first domain having site-specific DNA binding activity and at least a second domain having a protein lysine methyltransferase activity, wherein the first domain comprises the DNA binding domain of a pluripotency associated transcription factor and the second domain is capable of methylating an lysine residue located in the tail region of a histone H3. In a specific embodiment of the invention, the pluripotency associated transcription factor is selected from one of the group consisting of: Oct3, Oct4, nanog, sox2, c-myc and klf4. Optionally, the protein lysine methyltransferase activity of the second domain is directed towards the lysine residue is lysine 4 of histone H3 (H3K4). In one embodiment of the invention the protein lysine methyltransferase activity of ESET/SETDB1 or an orthologue or homologue thereof is utilised. As such, the second domain may comprise a protein lysine methyltransferase activity capable of mediating histone 3 lysine 9 tri-methylation (H3K9me3), comparable or identical to that catalysed by ESET/SETDB1.

A fifth aspect of the invention provides a method for identifying a modulator of pluripotency comprising exposing a library of candidate pluripotency modulating compounds to an ESET/SETDB1 polypeptide, identifying whether any of the candidate pluripotency modulating compounds bind to or inhibit the activity of the ESET/SETDB1 polypeptide, and identifying any candidate pluripotency modulating compounds that bind to or inhibit the activity of the ESET/SETDB1 polypeptide as a modulator of pluripotency. Suitably, the compound or molecule is selected from: a small molecule, an aptamer, a polypeptide, and oligopeptide, an oligonucleotide, a polyamine, an analogue of s-adenosyl-methionine, a substituted form of s-adenosyl-methionine, a nucleotide analogue, a nucleoside analogue, or an antibody or a fragment thereof. Optionally, the compound or molecule is either an inhibitor of ESET/SETDB1 catalytic activity, or a compound or molecule agonizes or promotes ESET/SETDB1 catalytic activity.

A sixth aspect of the invention provides for an inhibitor of ESET/SETDB1 activity or expression for use in the treatment of pluripotent cancer stem cells. Optionally, the cancer stem cells are selected from lung or breast cancer stem cells. In one embodiment of the invention, the inhibitor is selected from: an siRNA, an shRNA, an antisense oligonucleotide, or an antisense polynucleotide.

### DRAWINGS

The invention is illustrated in the following drawings in which:

FIG. 1 shows that Eset is required for normal ES cell phenotype. (A) Western blot showing down-regulation of ESET and H3K9me3 at day 3 (first lane) and day 4 (third lane) after Eset shRNA transfection. Tubulin and H3K4me2 served as loading controls. (B) Alkaline phosphatase staining of Eset shRNA and empty vector-transfected ES cells after 6 days of puromycin selection. (C) Morphology of FACS-sorted Eset knockdown cells and vector control cells after 4 days in ES culture medium. Scale bar represents 50 μm.

FIG. 2 shows Relative levels of gene expression in Eset knockdown cells at day 5 after transfection. Error bars: s.d. of three technical replicates.

FIG. 3 shows images of five representative colonies from three different wells of (top) vector control and (bottom) Eset knockdown ES cells after 4 days of culture in TS medium. Cdx2-positive cells are labeled in red. Nuclei are labeled in blue. Scale bar, 100 μm.

FIG. 4 shows (a) ChIP analysis of H3K9me3 on Cdx2 and Oct4 promoters in ES cells. ChIP primers C1 to C10 refers to the ChIP primers used to detect H3K9me3 on Cdx2 promoter. Primers O1 and O2 of Oct4 promoter served as negative control. (b) Carrier ChIP of H3K9me3 performed on FACS-sorted Eset knockdown ES cells. 293T cells were added as carrier. (c) Graph shows the relative levels of H3K9me3 on Cdx2 promoter and major satellite in Eset knockdown cells compared to vector control cells after normalizing against their respective input. Error bars, s.d. of two independent experiments.

FIG. 5 shows co-immunoprecipitation of ESET with Oct4. Expression vectors indicated were transfected in 293T cells and Flag-tagged Oct4 protein was immunoprecipitated. Immunoprecipitant (IP) and supernatant were subjected to Western blot (WB) with anti-HA (ESET, top panel) and anti-Flag (Oct4, bottom panel) antibodies. HA, haemagglutinin.

FIG. 6 shows (a) ES cell lysate were immunoprecipitated using anti-HA, anti-ESET (Abcam and Santa Cruz), anti-SUMO-1, anti-PML and anti-Oct4 antibodies and immunoblotted (WB) with antibodies indicated. (b) ES cell lysate were immunoprecipitated with the indicated antibodies in the presence or absence of NEM, a sumo isopeptidases inhibitor.

FIG. 7 shows (a) carrier ChIP of H3K9me3 performed on Zhbtc4 ES cells treated with tetracycline (Tc) for indicated days. (b) graph shows the relative levels of H3K9me3 on Cdx2 promoter and major satellite in Zhbtc4 ES cells treated with Tc compared to untreated cells after normalizing against their respective input. Error bars, s.d. of two independent experiments. (c) Western Blot showing down-regulation of ESET upon depletion of Oct4 at day 2 of Tc treatment of Zhbtc4 ES cells.

FIG. 8 shows immunostaining of (top) mES cells and (bottom) mEpiSC, both marked by Oct4 (green), shows that ESET (red) co-localizes with PML nuclear bodies (yellow) in mES cells but not mEpiSC. Feeder cell (arrow head) shows intense ESET foci that overlap with PML nuclear bodies. Scale bar, 10 μm.

FIG. 9 shows the amino acid alignment of murine ESET (“Query”) and its human orthologue SETDB1 (“Subject”).

FIG. 10 shows a histogram of GEO expression data for SETDB1 in human squamous lung cancer.

FIG. 11 shows a histogram of GEO expression data for SETDB1 in human breast cancer cell lines compared with normal mammary epithelium.

### DETAILED DESCRIPTION

All references cited herein are incorporated by reference in their entirety. Unless otherwise defined, all technical and scientific terms used herein have the same meaning as commonly understood by one of ordinary skill in the art to which this invention belongs. It will be understood that standard molecular biological techniques are used in carrying out of this invention. Such techniques are described, for example, in Sambrook J. et al, Molecular Cloning: a Laboratory Manual, Cold Spring Harbor Press, Cold Spring Harbor, N.Y.

Murine ESET (ERG-associated protein with SET domain; NM_{—}018877 (SEQ ID NOs: 1 and 2, cDNA and polypeptide respectively) and its human orthologue SETDB1 (SET domain bifurcated 1) which is known to exist in two alternatively spliced isoforms (isoform 1: NM_{—}001145415.1 (SEQ ID NOs: 3 and 4); isoform 2: NM_{—}012432 (SEQ ID NOs: 5 and 6) are histone methyltransferases that catalyze a repressive mark on euchromatin by mediating histone 3 lysine 9 tri-methylation (H3K9me3). Full-length ESET protein contains the tudor domain, methyl-CpG binding domain and a bifurcated SET domain that is responsible for its catalytic activity. Eset-null embryos die at peri-implantation stage with defective growth of the inner cell mass (ICM). However, the precise role of ESET in early development has remained unknown. In experiments described in detail below, the role of ESET in maintenance of a pluripotent state has been determined and surprisingly it has been found that ESET interacts directly with pluripotency associated transcription factors such as Oct4. For the avoidance of doubt the present invention utilises the terms ESET/SETDB1 interchangeably in reference to the H3K9me3 activity that regulates pluripotency in mammalian cells and is coordinated at least in part by the transcription factor Oct4. As used herein “SETDB1” refers to the human orthologue of the murine ESET and encompasses all isoforms, oligomers and variants of the protein, including post-translationally modified variants of SETDB1 (e.g. SUMOylated forms of SETDB1).

The catalytic ability of ESET to methylate H3K9 is described in WO-03/048352. However, the ability of ESET methyltransferase activity to regulate the pluripotent state or to form a complex with pluripotency associated transcription factors, such as Oct4, has not been identified previously.

The present invention also identifies an important epigenetic silencing mechanism that prevents pluripotent ES cells from differentiating into the trophectoderm lineage. This is despite the fact that ES cells can differentiate into all cell types of the body and yet possess a limited capacity to form trophectoderm cells. This unique epigenetic mechanism mediated by ESET/SETDB1, however does not seem to be present in murine EpiSC, which accounts for their unequal propensity to differentiate into trophectoderm cells. Notably, human ES cells share many characteristics of murine EpiSC, for example, its tendency to differentiate into trophectoderm cells. Thus, the mechanisms identified according to the present invention have important implications for elucidating some of the fundamental differences between mouse and human ES cells, specifically the correlation of epigenetic state and the commitment to trophectoderm lineage differentiation. More importantly, ESET/SETDB1, like Oct4, is a maternally inherited protein in the oocyte, and is critical for the establishment of pluripotent cells in the inner cell mass (ICM) and the trophectoderm lineage during preimplantation development, by the repression of Cdx2. This is consistent with the highly similar lack of ICM in both the ESET and Oct4 mutant blastocysts.

The present invention provides a clear demonstration of an epigenetic activity that is directly associated with transcription factors known to control pluripotency and may represent a key biological mechanism through which the pluripotent state is regulated.

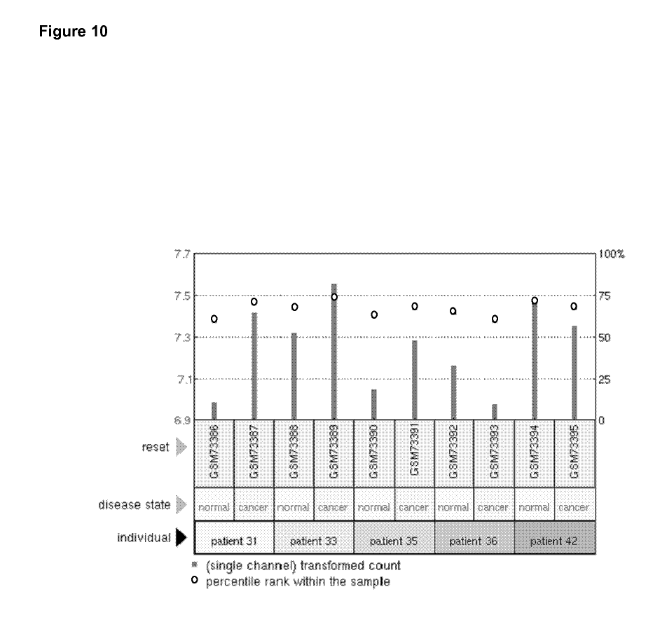

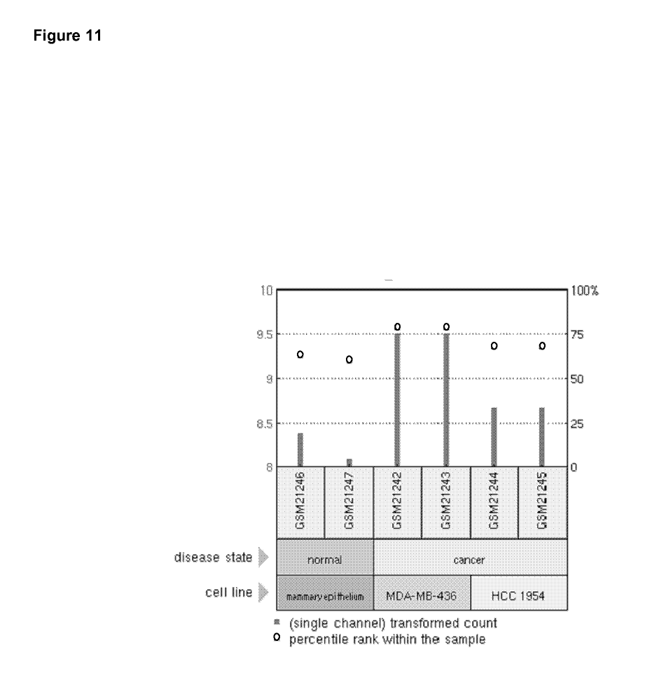

Another related area of utility for the present invention is in cancer therapy. Most if not all cancers undergo epigenetic changes, including significantly the down-regulation and silencing of tumour suppressor genes and the up-regulation of oncogenes. Reactivation of tumour suppressor genes can ameliorate cancer phenotype as can down-regulation of oncogenes. Hence, a method of controlling gene expression and cell fate decisions in vivo is a very promising avenue to cancer therapy. In the present invention, significantly elevated levels of SETDB1 expression is seen in tissue biopsies taken from human squamous lung cancer and breast cancer tumours (see FIGS. 10 and 11). However, given that ESET/SETDB1 expression is shown herein to be required for pluripotency, it is envisaged that it may be more highly expressed in sub-populations of cancer stem cells within the overall tumour. For this reason, ESET/SETDB1 might not appear to be expressed highly in many cancers as a whole, but could still play a crucial role in maintaining cellular self renewal in the subset of cancer stem cells within a tumour.

The term ‘cancer’ is used herein to denote a tissue or a cell located within a neoplasm or with properties associated with a neoplasm. Neoplasms typically possess characteristics that differentiate them from normal tissue and normal cells. Among such characteristics are included, but not limited to: a degree of anaplasia, changes in cell morphology, irregularity of shape, reduced cell adhesiveness, the ability to metastasise, increased levels of angiogenesis, increased cell invasiveness, reduced levels of cellular apoptosis and generally increased cell malignancy. Terms pertaining to and often synonymous with ‘cancer’ include sarcoma, carcinoma, tumour, epithelioma, leukaemia, lymphoma, polyp, transformation, neoplasm and the like.

The term ‘reprogramming’ as used herein, refers to the step of altering epigenetic modifications within the nucleus of a cell which results in the re-activation of pluripotent/stemness factors and/or the silencing of specific differentiation factors, and thus, mediating the induction of a pluripotent state. Reprogramming facilitates a reduction in cell fate commitment and, thus, the differentiation state of the cell as a whole and in particular the nucleus. In essence, reprogramming consists of returning a somatic differentiated or committed nucleus to a gene expression, epigenetic, and functional state characteristic of a pluripotent stem cell, such as an induced pluripotent stem cell (iPS cell), an embryonic stem cell (ES cell), an epiblast stem cell (EpiSC) or a primordial germ cell (PGC). Reprogramming of somatic cell nuclei is a preferred first step in procedures such as somatic cell nuclear transfer (SCNT), but is also of interest in other procedures where control of cell differentiation state—i.e. potency—is important. At present the definitive definition of what constitutes a pluripotent state can be unclear. Hence, the present invention provides a method for achieving a greater level of pluripotencyin cells that have been only partially reprogrammed or which may express certain genetic markers of pluripotency but have yet to adopt the appropriate morphology of a truly pluripotent cell, for instance by expressing ESET/SETDB1 in the cell.

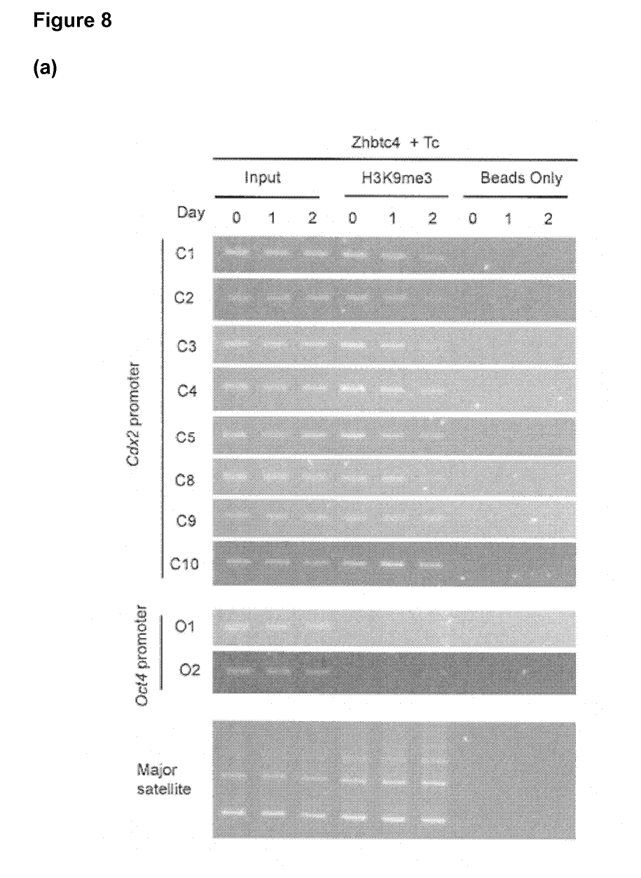

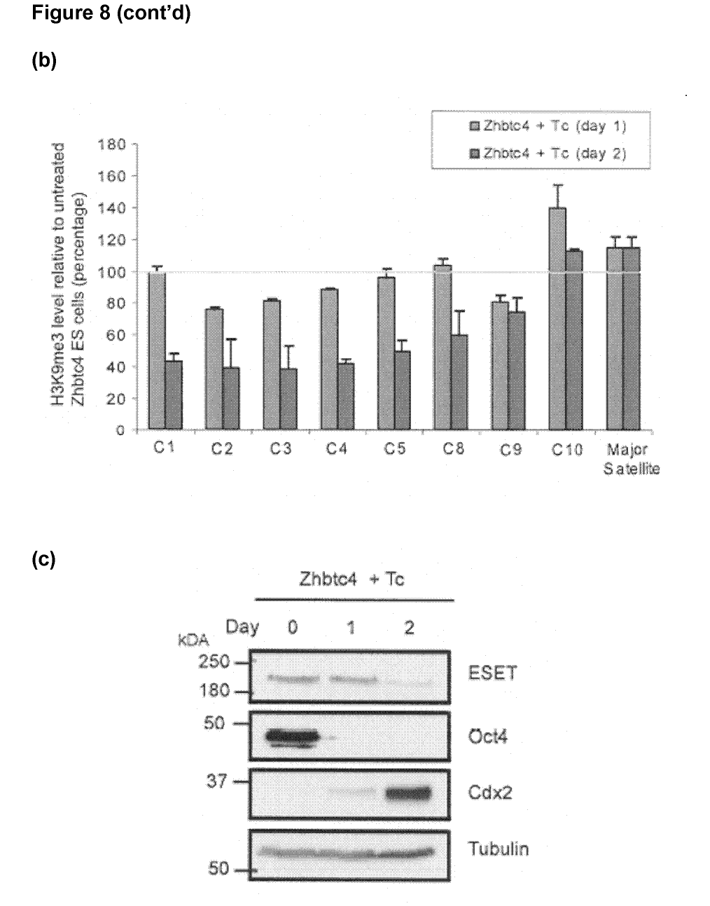

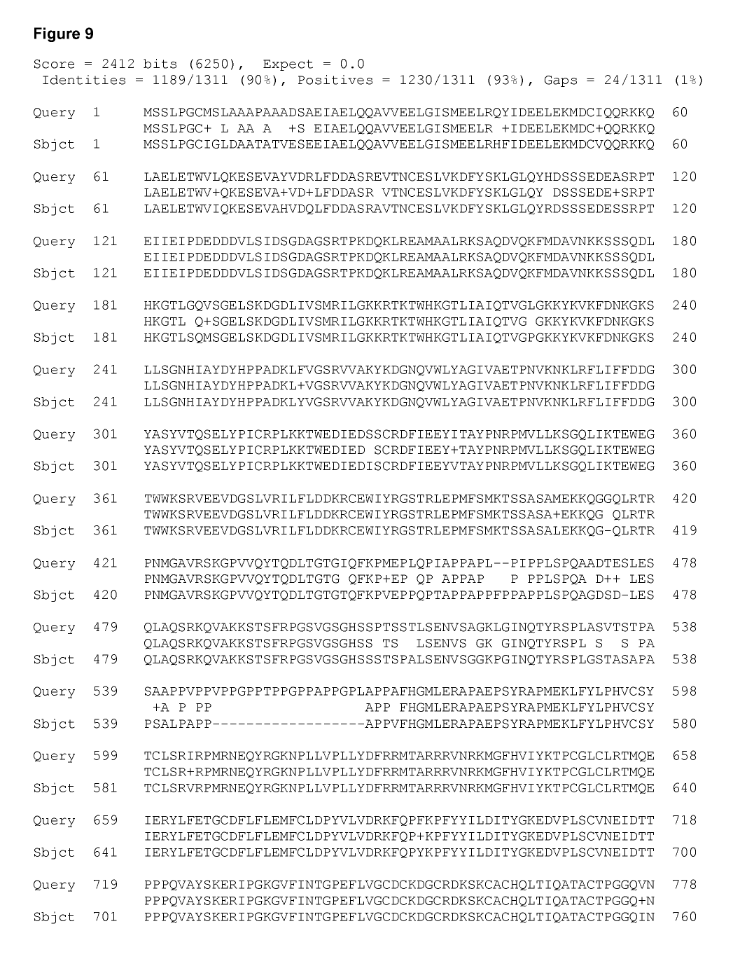

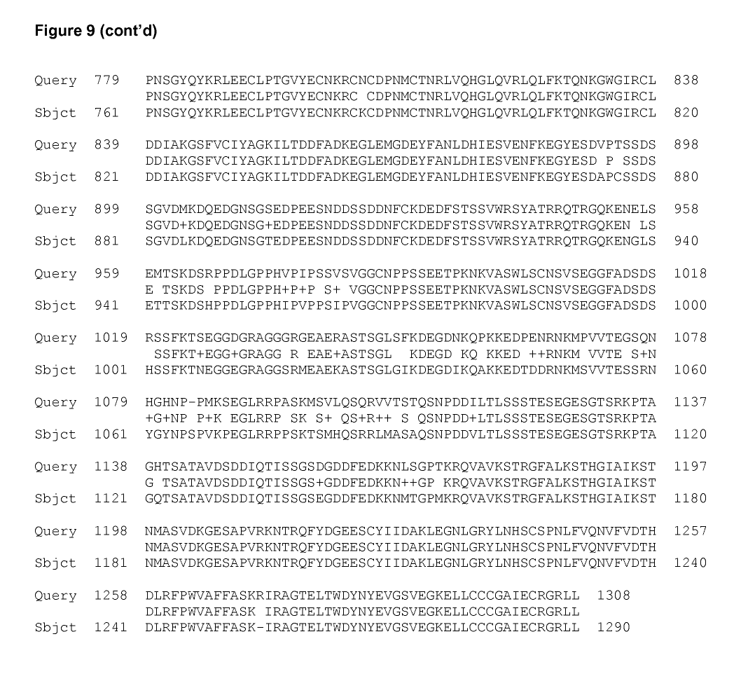

Derivatives and homologues of the ESET/SETDB1 sequences of the present invention are considered to include orthologues of the sequences from other species and mutants that nonetheless exhibit a high level of functional equivalence—i.e. the ability to interact with pluripotency associated transcription factors and thereby effect epigenetic modification of substrate proteins and polypeptides in vivo. Typically, derivatives, homologues and orthologues of ESET/SETDB1 will exhibit a substantially similar sequence identity—indeed, ESET and SETDB1 show 93% sequence identity (see FIG. 8). By substantially similar sequence identity, it is meant that the level of sequence similarity is from about 50%, 60%, 70%, 80%, 90%, 95% to about 99% identity. Percent sequence identity can be determined using conventional methods (Henikoff and Henikoff Proc. Natl. Acad. Sci. USA 1992; 89:10915, and Altschul et al. Nucleic Acids Res. 1997; 25:3389-3402). Alternatively, homologues of the polypeptides of the invention can be those sequences that are able to demonstrate the ability to hybridise with the sequences described herein, under conditions of high, medium or low stringency.

The term ‘expression vector’ is used to denote a DNA molecule that is either linear or circular, into which another DNA sequence fragment of appropriate size can be integrated. Such DNA fragment(s) can include additional segments that provide for transcription of a gene, such as ESET or SETDB1, encoded by the DNA sequence fragment. The additional segments can include and are not limited to: promoters, transcription terminators, enhancers, internal ribosome entry sites, untranslated regions, polyadenylation signals, selectable markers, origins of replication and such like. Expression vectors are often derived from plasmids, cosmids, viral vectors and yeast artificial chromosomes; vectors are often recombinant molecules containing DNA sequences from several sources. The expression vectors of the present invention may be maintained episomally or integrated into the genome of the host cell. Vectors that are suitable for random or non-targeted integration include lentiviral or retroviral expression vectors (Ye et al. Methods Mol Biol. (2008) 430:243-53; Brambrink et al. Cell Stem Cell. 2008 Feb. 7; 2(2):151-9). Expression vectors that achieve targeted integration into the genome of the host cell can also be used via a homologous recombination approach.

The term ‘operably linked’, when applied to DNA sequences, for example in an expression vector, indicates that the sequences are arranged so that they function cooperatively in order to achieve their intended purposes, i.e. a promoter sequence allows for initiation of transcription that proceeds through a linked coding sequence as far as the termination signal.

The term ‘isolated’, when applied to a polypeptide or complex of polypeptides, is a polypeptide that has been removed from its natural organism of origin. Suitably the isolated polypeptide is substantially free of other polypeptides native to the proteome of the originating organism. It is most preferred that the isolated polypeptide be in a form that is at least 95% pure, more preferably greater than 99% pure. In the present context, the term ‘isolated’ is intended to include the same polypeptide in alternative physical forms whether it is in the native form, denatured form, dimeric/multimeric, glycosylated, crystallised, or in derivatized forms. Reference to a ‘complex’ as used herein includes instances where the first and second polypeptide domains are comprised within a single polypeptide chain, also where the first and second domains are included within separate polypeptide chains that are non-covalently associated with each other, as well as where post translational covalent bonds are formed to link separate domains together into an associated functional unit.

Particular small nucleic acid molecules that are of use in the invention as inhibitors of ESET/SETDB1 are short stretches of double stranded RNA that are known as short interfering RNAs (siRNAs). These interfering RNA (RNAi) techniques allow for the selective inactivation of gene function in vivo. In the present invention, RNAi can be used to knock-down ESET/SETDB1 expression in cells. In this process, double stranded mRNAs are recognized and cleaved by the dicer RNase resulting in 21-23 nucleotide long stretches of RNAi. These RNAis are incorporated into and unwound by the RNA-inducing silencing complex (RISC). The single antisense strand then guides the RISC to mRNA containing the complementary sequence resulting in endonucleolytic cleavage of the mRNA (Elbashir et al. (2001) Nature 411; 494-498). Hence, this technique provides a means for the targeting and degradation of ESET/SETDB1 mRNA in cells when inhibition of a self-renewing pluripotent phenotype is desirable. Particular utility for RNAi targeted at ESET/SETDB1 expression can be found in the treatment of cancers, where therapy is intended for treatment of cancer stem cell progenitors.

Commercially available short hairpin RNAs (shRNAs) that are suitable for use in RNAi and which specifically target SETDB1 are set out below (Sigma, Poole, Dorset, UK):

| | |
|---|---|
| SEQ ID NO: 7 | CCGGGCCTTGATCTTCCATGTCATTCTCG |
| | AGAATGACATGGAAGATCAAGGCTTTTTG |
| | Clone ID: NM_018877.2-4384s1c1 |
| | Accession Number(s): NM_018877.2 |
| | Region: 3UTR |
| SEQ ID NO: 8 | CCGGCCCATGAGAAACGAACAGTATCTCG |
| | AGATACTGTTCGTTTCTCATGGGTTTTTG |
| | Clone ID: NM_018877.2-1934s1c1 |
| | Accession Number(s): NM_018877.2 |
| | Region: CDS |
| SEQ ID NO: 9 | CCGGCCCGAGGCTTTGCTCTTAAATCTCG |
| | AGATTTAAGAGCAAAGCCTCGGGTTTTTG |
| | Clone ID: NM_018877.2-3660s1c1 |
| | Accession Number(s): NM_018877.2 |
| | Region: CDS |
| SEQ ID NO: 10 | CCGGCCACATTGAAAGTGTGGAGAACTCG |
| | AGTTCTCCACACTTTCAATGTGGTTTTTG |
| | Clone ID: NM_018877.2-2746s1c1 |
| | Accession Number(s): NM_018877.2 |
| | Region: CDS |
| SEQ ID NO: 11 | CCGGCCAGACATATCGGTCACCTTTCTCG |
| | AGAAAGGTGACCGATATGTCTGGTTTTTG |
| | Clone ID: NM_018877.2-1687s1c1 |
| | Accession Number(s): NM_018877.2 |
| | Region: CDS |

Screening of molecules and proteins for binding to ESET/SETDB1, ESET/SETDB1-Oct4 or ESET/SETDB1-Nanog complexes can be performed via automated high-throughput screening procedures. Hence, the invention provides methods for identifying ESET/SETDB1 interacting molecules via detection of a positive binding interaction between the ESET/SETDB1 and a target molecule. Further screening steps may be used to determine whether the identified positive binding interaction is of pharmacological importance—i.e. whether the target molecule is capable of moderating ESET/SETDB1 biological activity or function. Moderation of activity may include inhibiting or agonizing the activity of the ESET/SETDB1 molecule. Inhibition of activity may be through competitive or non-competitive inhibition. If a molecule with a positive moderating effect is identified, the molecule is classified as a ‘hit’ and can then be assessed as a potential candidate drug. Additional factors may be taken into consideration at this time or before, such as the absorption, distribution, metabolism and excretion (ADME), bio-availability and toxicity profiles of the molecule, for example. If the potential drug molecule satisfies the pharmacological requirements it is deemed to be pharmaceutically compatible. Suitable compositions can be formulated for testing the activity in-vitro and in-vivo in accordance with standard procedures known in the art.

In accordance with the invention assays can be developed to facilitate high throughput screening of candidate compounds in order to identify modulators of ESET/SETDB1 activity, for particular use in modulating the pluripotent state in target cells and cell types. In one such exemplary assay, wells of a multi-well plate are coated with an appropriate immobilised substrate, such as an assembled recombinant nucleosomes or a histone peptide (preferably including an H3K9-comprising target peptide for ESET/SETDB1) immobilised via biotin-streptavidin linkage. To each well is added a reaction solution comprising ESET/SETDB1, S-adenosyl-methionine co-factor and one of a library of candidate modulator molecules. If the candidate modulator molecule acts as an inhibitor of ESET/SETDB1 then methylation of amino acid residues on histone H3 (comprised within the nucleosome substrate) or on the H3 peptide can be reduced or prevented. The determination of methylation status of the lysine residues in the histone H3/peptide can be determined by use of an antibody that specifically binds to the methylated target lysine residue in histone H3 (i.e. H3K9me3). The antibody can be linked to a colour generating reaction, so as to form an ELISA-type assay. In this way, wells of the multi-well plate that show a colour reaction correspond to reactions where inhibition of ESET/SETDB1 has not occurred, whereas candidate compounds present in the uncoloured wells are identified as candidate inhibitors of ESET/SETDB1 activity.

Alternative candidate molecule screens can be devised that are directed towards correlation of reporter gene expression with methylation status of amino aid residues in histone substrates comprised within nucleosomes located in the promoter region of the reporter gene construct. Reporter gene expression can be switched on or off depending upon whether the methylation catalysed by ESET/SETDB1 initiates or represses gene expression. Performing the reporter assay in the presence of a candidate modulator compound allows for determination of whether the modulator exhibits an agonistic or antagonistic effect on ESET/SETDB1 activity.

The invention is further described in the following non-limiting examples.

### EXAMPLES

### Example 1

Mouse ES cells were transfected with vector expressing short-hairpin RNA (shRNA) sequences to knockdown ESET. The vectors also contained EGFP and puromycin-resistance selection markers. The methodology was as follows. Short-hairpin RNA (shRNA) was cloned into the Bglll and HindIII sites of the pSuper.puro vector (Oligoengine). Sequences for shRNA are:

(SEQ ID NO: 12)
5′ GATCCCCGATGTGAGTGGATATATCGTTCAAGAGACGATATATCCA
CTCACATCTTTTTA 3′
and
(SEQ ID NO: 13)
5′ AGCTTAAAAAGATGTGAGTGGATATATCGTCTCTTGAACGATATAT
CCACTCACATCGGG 3′

For construction of shRNA-pIRES-EGFP, pSuper.puro with or without shRNA insert that has been digested with NotI and HincII were ligated to pIRES-EGFP (Clontech) that has been digested with NruI and NotI. For transfection, 0.5×10⁶ ES cells plated on a 6-well dish overnight were transfected with 1 μg of plasmid using Lipofectamine reagent (Invitrogen). Transfected cells were selected with 1 μg/ml puromycin (Sigma) 24 hours after transfection and passaged at equal ratio upon reaching confluency.

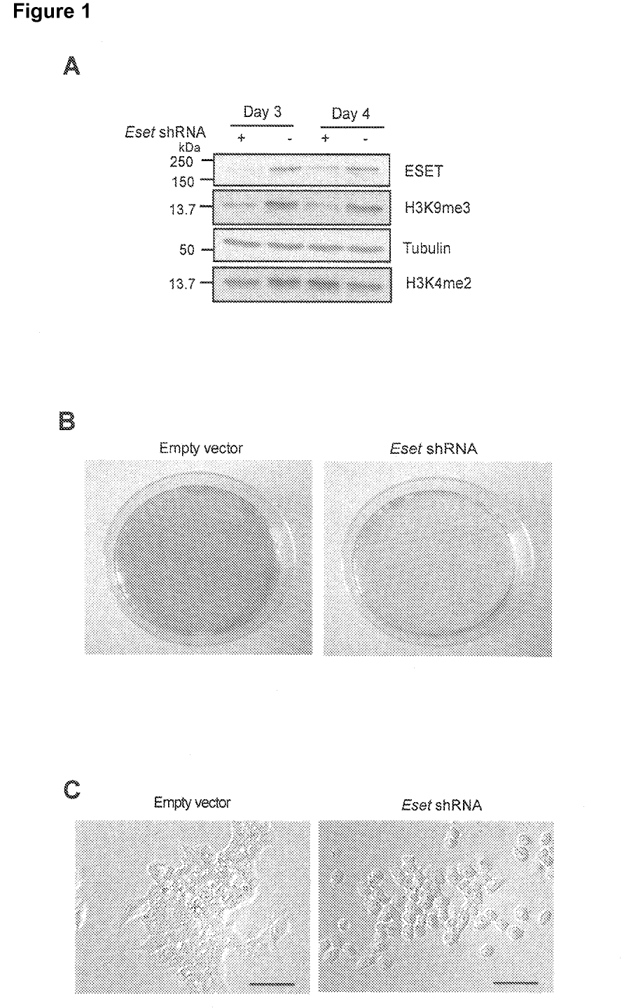

Knockdown of gene expression was confirmed using western blotting (FIG. 1A). Cell lysate was extracted using cold RIPA buffer consisting 50 mM Tris pH 8.0, 150 mM NaCl, 1% NP-40, 0.5% sodium deoxycholate and 0.1% SDS added with protease inhibitor (Roche) for 30 minutes on ice, followed by centrifugation at 13k rpm. Supernatant was collected and protein concentration measured by Bradford assay (Sigma). Total protein (20-30 μg) was separated by Tris-glycine SDS polyacrylamide gel and transferred to Hybond-P PVDF (Amersham) membrane. Primary antibodies used were ESET (Upstate), α-tubulin (Sigma), H3K9me3 (Upstate), H3K4me2 (Abcam) and Oct-3 (BD). Proteins on polyacrylamide gel were visualized by staining with Imperial Protein Stain (Pierce).

A general visualisation of ES cell numbers was obtained by alkaline phosphatase staining using standard reagents and protocols from Sigma (Poole, Dorset). After six days of puromycin selection, the numbers of ES cells were decreased as judged by alkaline phosphatase activity (FIG. 1B).

To investigate further the morphology of the knockdown cells, transfected cells were FACS sorted on the basis of EGFP expression 24 hours post-transfection and then cultures for two days in ES cell medium. Essentially, the cells were cultured without feeders on gelatin-coated culture dish in Dulbecco's modified Eagle's medium/F12 nutrient mixture without L-glutamine (DMEM/F12) (GIBCO) supplemented with 20% fetal bovine serum (GIBCO), 2 mM L-glutamine (GIBCO), 0.1 mM MEM non-essential amino acids (GIBCO), 100 U/ml Penicillin/Streptomycin (GIBCO), 1 mM sodium pyruvate (Sigma), 0.12% sodium bicarbonate solution (Sigma), 50 μM 2-mercaptoethanol (GIBCO), 0.15 mM of each nucleoside comprising adenosine, cytidine, guanosine and uridine and 0.05 mM of tymidine (Sigma) and 2000 μml leukemia inhibitory factor (Chemicon).

The morphology of the knockdown cells was clearly different from the ES cells transfected with the empty vectors, suggesting that ESET is important for the self-renewal and maintenance of a normal pluripotent phenotype (FIG. 1C).

### Example 2

Quantitative real-time RT-PCR was used to assess the RNA levels of several candidate transcripts in control and ESET knockdown cells. Cells were harvested without FACS sorting and RNA was prepared using RNeasy Mini Kit (Qiagen) and cDNA was synthesized from 1 μg of RNA using SuperScript™ III reverse transcriptase (Invitrogen). Endogenous mRNA levels were measured by real-time PCR based on SYBR Green detection with the ABI Prism 7000 real-time PCR machine (Applied Biosystem). Each reaction in a total volume of 20 μl contained 1 μl of cDNA that was diluted ten times, 1 μM of forward and reverse primer and 1× QuantiTect SYBR Green Master Mix reagent (Qiagen). Standard curve for each primers were performed in the same sample plate to determine the relative quantification of the transcript. Real-time PCR was done in triplicates and normalized with GAPDH, a house-keeping gene. The data were then normalized against vector control which was defined as 1.0.

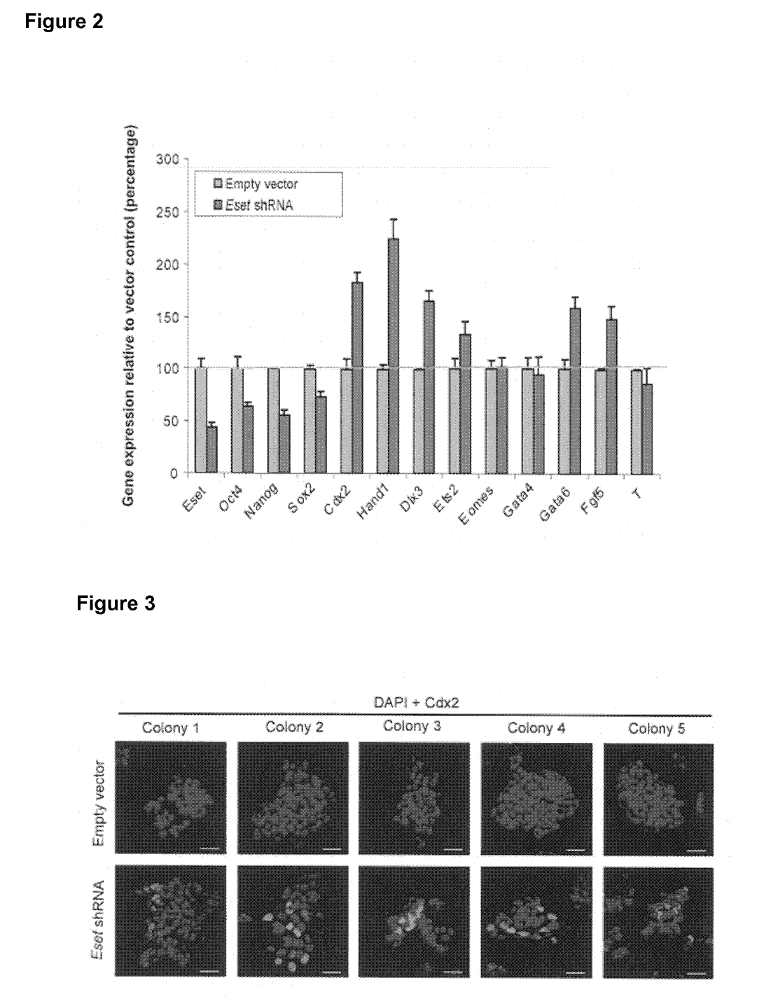

As shown in FIG. 2, down-regulation of ESET was accompanied by down-regulation of pluripotency marker genes (Oct4, Nanog and Sox2) and up-regulation of differentiation markers (Cdx2, Hand1, Dlx3, Ets2, FgfS and Gata6). The up-regulation of trophectoderm markers such as Cdx2, Hand1, Dlx3 and Ets2 after ESET down-regulation is of particular interest as these genes are not normally induced when ES cells undergo differentiation. This indicates that ESET is particularly important for maintaining pluripotency by suppressing expression of trophectoderm-specific genes.

To explore the trophectoderm aspects in greater depth, ES cells that were transfected with either the ESET shRNA or empty vectors were FACS-sorted three days post-transfection and then cultured in a medium conducive to development of trophectoderm cells (TS medium—described in Takeda, 1998). After four days in TS medium Cdx2 positive cells were observed in at least 50% of ESET knockdown colonies, whereas expression of the same gene in cells transfected with the empty vector was practically non-existent (FIG. 3).

### Example 3

ESET depletion for the ES cells led to up-regulation of Cdx2. A catalytic function of ESET is trimethylation of histone H3K9 (H3K9me3) and it was therefore postulated that downregulation of ESET could result in decreased levels of H3K9me3 at the Cdx2 promoter. Chromatin immunoprecipitation (ChIP) was used to assess the normal levels of this epigenetic modification at the Cdx2 promoter and any changes when ESET expression was disrupted.

Chromatin immunoprecipitation was performed according to published protocol (Lee et al., 2006b) with some modifications. Briefly, cells were crosslinked with 1/10 volume of fresh 11% formaldehyde solution for 10 minutes and quenched with 1/20 volume of 2.5 M glycine. Cells were sonicated to an average of 500 bp and immunoprecipitated overnight with antibody that was pre-incubated with 100 μl Dynabeads M-280 Sheep Anti-Rabbit (overnight). For isolation of DNA, 100 ul of 10% Chelex (w/v) was added to washed beads, vortexed and boiled for 10 minutes (Nelson et al., 2006). After cooling to room temperature, 100 μg/ml of proteinase K was added and beads were incubated for 30 min at 55° C. while shaking. Beads were boiled for another 10 minutes, centrifuged and supernatant collected. The Chelex/bead fraction is vortexed with another 100 μl of water, centrifuged and the supernatant collected is combined with the first supernatant. About 2 to 3 μl of DNA were used as template for PCR amplification with Red Taq (Sigma). For carrier ChIP, 3×10⁷ 293T cells were added to 1×10⁶ FACS sorted, GFP positive ES cells that was transfected with either ESET-shRNA or empty vector for three days (selected with puromycin for two days).

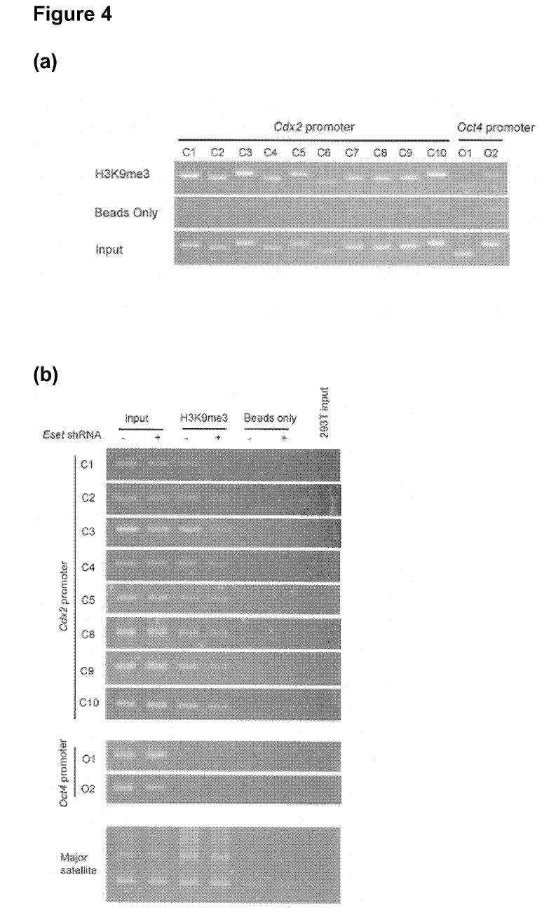

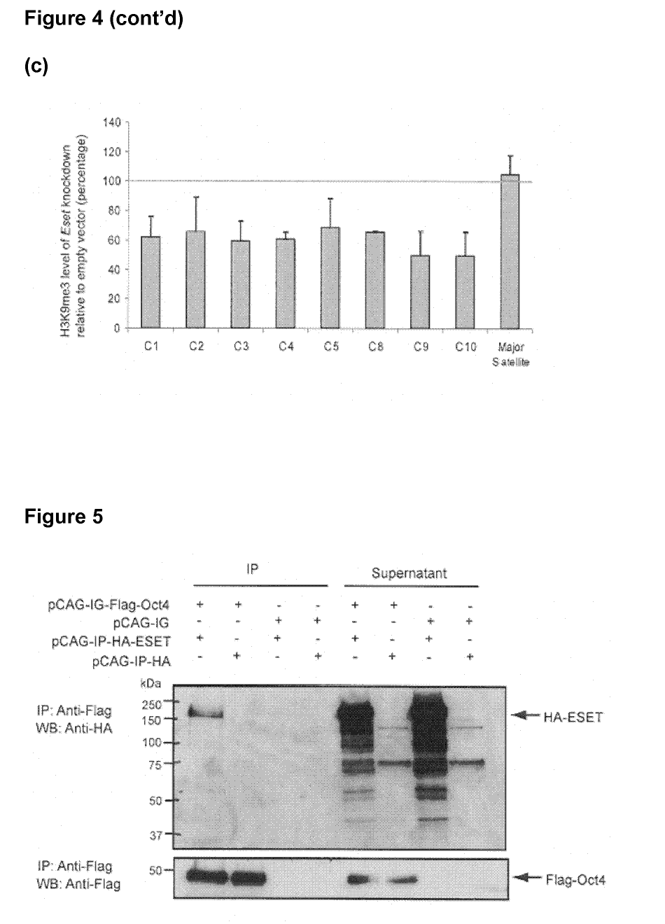

FIG. 4A shows that in normal ES cells the H3K9me3 mark is found at the Cdx2 promoter, but not at the Oct4 promoter (consistent with expression of these genes being repressed and active in ES cells respectively). FIG. 4B demonstrates that in the ESET-depleted cells there is decreased H3K9me3 at the Cdx2 promoter. Data from two independent ESET knockdown experiments, showing average down-regulation of H3K9me3 at the Cdx2 promoter of 50%-60% are shown in FIG. 4C.

Collectively these results demonstrate that ESET-mediated H3K9me3 represses Cdx2 expression in pluripotent cells.

### Example 4

Depletion of ESET and Oct4 have a similar effect i.e. commitment to the trophoectoderm cell fate, and Cdx2 is also a transcriptional repression target of Oct4. In order to test the hypothesis that ESET and Oct4 might co-operate, double-transfectant HEK-293T cells were created which contained expression vectors for FLAG-Oct4 and HA-ESET For co-transfection experiment, 3.5×106 293T cells plated on 10 cm2 dish overnight were transfected with 18 ug of DNA comprising 9 ug of two constructs with Lipofectamine reagent (Invitrogen).

To test for an interaction between ESET and Oct4, proteins from the double transfectants were immunoprecipitated. Briefly, Cells were harvested for immunoprecipitation 48 hours after transfection. Cells from 10 cm 2 dish were washed twice in cold PBS and scraped into 1 ml of PBS. Cell pellet were resuspended in 200 μl of immunoprecipitation buffer (50 mM Tris pH 8.0, 150 mM NaCl and 1% NP-40 added with protease inhibitor from Roche), incubated on ice with occasional tapping for 30 minutes and centrifuged at 13k rpm for 30 minutes at 4° C. 100 μl of the supernatant were then diluted with 900 ul dilution buffer (50 mM Tris pH 8.0 and 150 mM NaCl added with protease inhibitor from Roche) so that the final concentration of the immunoprecipitation reaction is 0.1% NP-40. Cell lysate was pre-cleared with 50 μl of 50% Protein A/G (Amersham) slurry for 1 hour at 4° C. Cell lysate was then centrifuged at 13k rpm for 20 minutes at 4° C. and 1 μl of Anti-HA antibody (Abcam ab9110) or 1 μl of Anti-Flag (Sigma F3165) was incubated with 900 μl of the supernatant at 4° C. overnight. Precipitation was performed by adding 50 μl of 50% Protein NG slurry to the reaction for 1 hour followed by 5 washes in buffer containing 50 mM Tris pH 8.0, 150 mM NaCl and 0.1% NP-40. Beads were boiled for 5 minutes with 50 μl 2× sample Laemlli buffer (Biorad) and 20 μl were loaded onto Tris-Glycine SDS Polyacrylamide gel.

As shown in FIG. 5, HA-ESET co-immunoprecipitated with FLAG-Oct4. This indicates that ESET physically interacts in vivo with the pluripotency associated transcription factor Oct4.

### Example 5

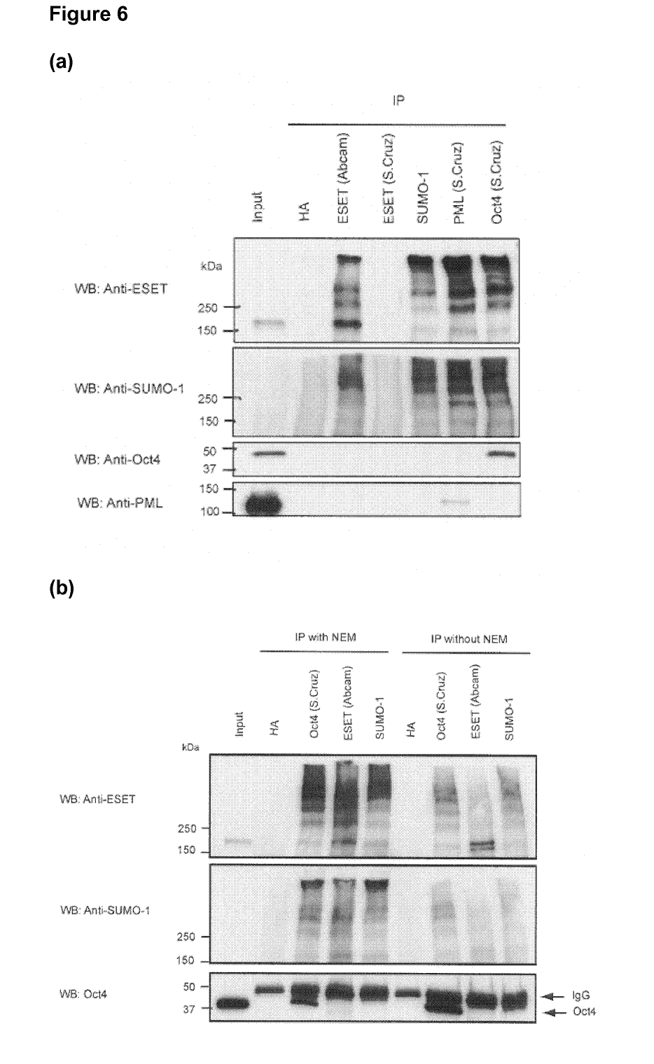

A similar experiment to that in Example 4 was performed using ES cells and the endogenous proteins. This demonstrated interaction of Oct4 with not just the expected 180 kDA ESET protein, but with several other ESET proteins of higher molecular weight (FIG. 6A, last lane).

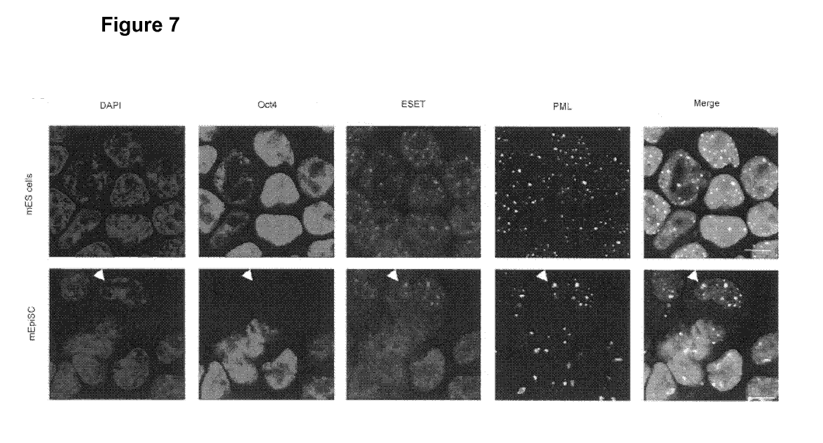

Indications of the possible source of these additional ESET candidates were derived from data on the intracellular expression patterns of ESET in ES cells. Immunostaining demonstrated that ESET is found in punctate foci at euchromatin regions, a pattern which overlaps with promyelocytic leukaemia (PML) bodies, as shown in FIG. 7. PML bodies are frequently sites of protein SUMOylation and hence the proteins isolated as described in the paragraph above were probed with antibodies to detect SUMO. This confirmed that in the ES cells ESET exists in a variety of SUMOylated forms (FIG. 6A). As would be predicted, these bands were less prominent when the immunoprecipitation was performed in the absence of N-ethylmaleimide (NEM), an inhibitor of SUMO isopeptidases (FIG. 6B).

### Example 6

To clarify further the functional interactions of Oct4 and ESET, Zhbtc4 ES cells that contain two tetracycline-regulatable Oct4 alleles were used. FIG. 7A-7C demonstrates that Oct4 depletion leads to decreased H3K9me3 at the Cdx2 promoter and decreased ESET expression. The same experiments were performed on major satellite DNA form carrier material to demonstrate that the altered H3K9me3 levels at the Cdx2 promoter was a specific, rather than a genome-wide effect.

This experiment in combination with the other examples provided herein demonstrate that the complex formed between ESET and Oct4 is capable of epigenetic regulation of the expression pro-differentiation gene, Cdx2. As such, this presents a paradigm for Oct4 mediated expression regulation of target genes and the maintenance of the pluripotent state.

In conclusion, an important epigenetic mechanism that maintains pluripotency by preventing differentiation of ES cells, notably into trophectoderm cells, has been identified. The Oct4-ESET mediated H3K9me3 epigenetic modification involved in the repression of Cdx2 may affect other genes, including Gata6, to underpin pluripotency. The synergistic action of Oct4 and ESET could also explain the previously described observation that Oct4 regulates expression of Cdx2. The sumoylation interacting motif (SIM) of Oct4 is apparently important in mediating the interaction of ESET and Oct4. Since ESET, like Oct4 is a maternally inherited protein in the oocyte, the Oct4-ESET may also be critical for the establishment of pluripotent cells in the inner cell mass (ICM), at least in part through repression of Cdx2. Notably, the loss of ESET or Oct4 results in the loss of the pluripotent ICM. The ESET-Oct4 interaction (and possibly also SUMOylated ESET-Oct4) is pivotal for both the establishment of pluripotency in the ICM and the maintenance of the pluripotent phenotype as a whole.

### Example 7

The BLAST algorithm was used to align the amino acid sequences of the murine ESET protein and its human orthologue, SETDB1. Standard settings were used, filters were off. The results are shown in FIG. 8. The two proteins are 90% identical and 93% homologous.

### Example 8

The GEO gene expression database (http://www.ncbi.nlm.nih.gov/geo/) was searched using the term “setdb1 homo sapiens cancer”. FIG. 10 demonstrates that there is a strong tendency for the human ESET orthologue to be up-regulated in squamous lung cancer. The gene is also up-regulated in human breast cancer cell lines compared with normal mammary epithelium (FIG. 11).

Although particular embodiments of the invention have been disclosed herein in detail, this has been done by way of example and for the purposes of illustration only. The aforementioned embodiments are not intended to be limiting with respect to the scope of the appended claims, which follow. It is contemplated by the inventors that various substitutions, alterations, and modifications may be made to the invention without departing from the spirit and scope of the invention as defined by the claims.

1. A method for controlling the pluripotent phenotype of a cell comprising modulating the expression or activity of an ESET/SETDB1 polypeptide, or a homologue thereof, within the cell.

2. The method of claim 1, wherein activity of the ESET/SETDB1 polypeptide is modulated by exposing the cell to a compound or molecule that modulates the catalytic activity of ESET/SETDB1.

3. The method of claim 2, wherein the compound or molecule is selected from the group consisting of: a small molecule; an aptamer; a polypeptide; and oligopeptide; an oligonucleotide; a polynucleotide; a polyamine; an analogue of s-adenosyl-methionine; a substituted form of s-adenosyl-methionine; a nucleotide analogue; a nucleoside analogue; and an antibody or a fragment thereof.

4. The method of claim 2, wherein the compound or molecule is an inhibitor of ESET/SETDB1 catalytic activity.

5. The method of claim 2, wherein the compound or molecule agonises or promotes ESET/SETDB1 catalytic activity.

6. The method of claim 1, wherein an ESET/SETDB1 encoding polynucleotide is introduced into the cell and expressed via a heterologous expression vector.

7. The method of claim 6, wherein the heterologous expression vector is an episomal vector.

8. The method of claim 7, wherein the heterologous expression vector comprises a nucleic acid sequence that encodes an ESET/SETDB1 polypeptide that is operatively linked to a promoter sequence.

9-11. (canceled)

12. The method of claim 6, wherein the heterologous expression vector integrates into the genome of the cell via homologous recombination.

13-14. (canceled)

15. The method of claim 1, wherein expression of the ESET/SETDB1 polypeptide is modulated by exposing the cell to a compound selected from the group consisting of: an siRNA; an shRNA; an antisense oligonucleotide; and an antisense polynucleotide.

16. The method of claim 1, wherein the compound comprises an shRNA selected from one or more of SEQ ID NOs: 7-13.

17. A pluripotent mammalian cell comprising a heterologous expression vector that encodes an ESET/SETDB1 polypeptide, or a homologue or derivative thereof.

18. The cell of claim 17, wherein the heterologous expression vector is integrated into the genome of the cell via homologous recombination.

19. The cell of claim 17, wherein the heterologous expression vector is episomally maintained.

20. The cell of claim 17, wherein the cell is derived from one of the group consisting of: a somatic cell; a multipotent stem cell; a unipotent stem cell; a cancer cell; a cancer cell line cell; a primordial germ cell; and a pluripotent cell.

21-22. (canceled)

23. A culture vessel comprising a culture of pluripotent mammalian stem cells according to claim 17, and a culture medium suitable for maintaining the pluripotent stem cells.

24. A method for reprogramming a somatic cell nucleus comprising expressing ESET/SETDB1 polypeptide, or homologue thereof, in a somatic cell that comprises the nucleus in combination with one or more pluripotency associated transcription factors.

25. The method of claim 24, wherein the pluripotency associated transcription factor is selected from one or more of the group consisting of Oct3, Oct4, nanog, sox2, c-myc, Dppa3, Dppa4 and klf4.

26. The method of claim 24, wherein the somatic cell nucleus is obtained from a cell selected from: a multipotent stem cell, a unipotent stem cell, a germ cell and a terminally differentiated cell.

27. The method of claim 24, wherein the cell is a human cell.

28. The method of claim 24, further comprising exposing the cell to an inhibitor of the MEK/ERK signalling pathway.

29-34. (canceled)

35. A method for identifying a modulator of pluripotency comprising exposing a library of candidate pluripotency modulating compounds to an ESET/SETDB1 polypeptide, identifying whether any of the candidate pluripotency modulating compounds bind to or inhibit the activity of the ESET/SETDB1 polypeptide, and identifying any candidate pluripotency modulating compounds that bind to or inhibit the activity of the ESET/SETDB1 polypeptide as a modulator of pluripotency.

36. The method of claim 35, wherein the compound or molecule is selected from: a small molecule, an aptamer, a polypeptide, and oligopeptide, an oligonucleotide, a polyamine, an analogue of s-adenosyl-methionine, a substituted form of s-adenosyl-methionine, a nucleotide analogue, a nucleoside analogue, or an antibody or a fragment thereof.

37. The method of claim 35, wherein the compound or molecule is an inhibitor of ESET/SETDB1 catalytic activity.

38. The method of claim 35, wherein the compound or molecule agonises or promotes ESET/SETDB1 catalytic activity.

39. An inhibitor of ESET/SETDB1 activity or expression for use in the treatment of pluripotent cancer stem cells.

40. The inhibitor of claim 39, wherein the cancer stem cells are selected from lung or breast cancer stem cells.

41. The inhibitor of claim 39, wherein the inhibitor is selected from the group consisting of: an siRNA; an shRNA; an antisense oligonucleotide; and an antisense polynucleotide.

42. The inhibitor of claim 41, wherein the inhibitor comprises an shRNA selected from one or more of SEQ ID NOs: 7-13.
